(19) United States
(12) Patent Application Publication
(10) Pub. No.: US 2022/0146383 A1
(43) Pub. Date: May 12, 2022

(54) MICROFLUIDIC SAMPLE LABELING

(71) Applicant: Hewlett-Packard Development Company, L.P., Spring, TX (US)

(72) Inventors: Alexander Govyadinov, Corvallis, OR (US); Viktor Shkolnikov, Palo Alto, CA (US)

(73) Assignee: Hewlett-Packard Development Company, L.P., Spring, TX (US)

(21) Appl. No.: 17/417,811

(22) PCT Filed: Jul. 26, 2019
(86) PCT No.: PCT/US2019/043556

§ 371 Date: Jun. 24, 2021

(51) Int. Cl. G01N 1/38; B01L 3/00; C12Q 1/6825; G01N 33/58

(52) U.S. Cl. CPC G01N 1/38; B01L 3/502784; B01L 3/502761; G01N 2001/388; C12Q 1/6825; G01N 33/582; B01L 2300/0829; B01L 3/502715

(57) ABSTRACT

The present disclosure is drawn to a method of labeling and identifying microfluidic samples. The method can include tagging a first microfluidic sample with a first combination of markers to obtain a first labeled sample; tagging a second microfluidic sample with a second combination of markers that is different than the first combination of markers to obtain a second labeled sample; introducing a common variable to the first labeled sample and the second labeled sample. The common variable can generates a first interaction with the first labeled sample that is different than a second interaction or lack of interaction with the second labeled sample. The method can further include based on the first interaction, identifying the first labeled sample by assaying for the first combination of markers.

### BACKGROUND

Microfluidic droplet based biological assays can exploit the chemical properties and the physical properties of fluids on a microscale. Microfluidic droplets can exhibit laminar flow without exhibiting turbulent flow, which can permit high control, increased diffusion rates, and increased reaction rates. Further, performing an assay on a microscale can decrease reagent associated costs. Accordingly, microfluidic droplet based assays are increasing in popularity in research, medical, and forensic applications, to name a few.

### BRIEF DESCRIPTION OF THE DRAWINGS

FIG. 1 is a flow diagram illustrating an example method of labeling and identifying individual microfluidic samples in accordance with the present disclosure;

FIG. 2 graphically illustrates a schematic view of an example a system for independently labeling partitioned microfluidic samples for identification in accordance with the present disclosure;

FIG. 3 graphically illustrates a schematic view of an example microfluidic partition network fluidly connected to a marker ingress network in accordance with the present disclosure;

FIG. 4 graphically illustrates a schematic view of an example a system for independently labeling partitioned microfluidic samples for identification in accordance with the present disclosure;

FIG. 5 graphically illustrates a schematic cross-sectional view of a microfluidic channel in accordance with the present disclosure;

FIG. 6 graphically illustrates a system for identifying an individual microfluidic sample from multiple partitioned microfluidic samples in accordance with the present disclosure;

FIG. 7 graphically illustrates an example electrochemical detection system in accordance with the present disclosure; and

FIG. 8 graphically illustrates an example electrochemical detection system in accordance with the present disclosure.

### DETAILED DESCRIPTION

Microfluidic droplets can act as micro-reactors and can allow for millions of simultaneous reactions to occur in parallel. In addition, these reactions can permit absolute quantitation of the target molecule present. For example, droplet based polymerase chain reaction (PCR) can allow for the quantification of the amount of nucleic acid molecules present in a sample. However, with no ability to identify one sample fluid from another, the application of conducting millions of simultaneous reactions in a bulk reactor is limited to a single sample fluid. Accordingly, a method for labeling and subsequently identifying individual microfluidic samples that can permit processing of multiple sample fluids simultaneously can improve diagnostic efficiency.

In accordance with this example and others, the present disclosure is drawn to a method of labeling and identifying individual microfluidic samples. The method includes tagging a first microfluidic sample with a first combination of markers to obtain a first labeled sample and tagging a second microfluidic sample with a second combination of markers that is different than the first combination of markers to obtain a second labeled sample. The method further includes introducing a common variable to the first labeled sample and the second labeled sample, wherein the common variable generates a first interaction with the first labeled sample that is different than a second interaction or lack of interaction with the second labeled sample. In further detail, the method includes, based on the first interaction, identifying the first labeled sample by assaying for the first combination of markers. In one example, the tagging of the first microfluidic sample with the first combination of markers includes admixing from 2 μL to 10 nL per individual marker into a fluidic volume from 1 μL to 10 μL of the first microfluidic sample. In another example, a marker in the first combination of markers and the second combination of markers includes individual markers selected from a redox marker, a fluorescing chemical, a magnetic marker, an antigen, a fluorescent antigen, phosphorescent, chemiluminescent, or a combination thereof. In yet another example, the first combination of markers and the second combination of markers are selected from a bank of markers that are independently identifiable relative to one another when present in the first labeled sample or the second labeled sample, and the bank of markers provide for combinations of markers generated from 4 to 128 possible combinations of markers. In a further example, the first labeled sample, the second labeled sample, or both are partitioned into multiple 3 μL to 1 μL partitioned volumes prior to the introducing of the common variable to individual partitioned volumes. In one example, the method further includes individually dispensing the partitioned volumes into individual wells of a well plate or individual channels of a microfluidic device either before or after introducing the common variable to the first labeled sample and the second labeled sample. In another example, the method further includes individually dispensing the partitioned volumes into a microfluidic device with fluidic blanks providing separation between partitioned volumes. In yet another example, the common variable includes reactants for polymerase chain reaction, enzyme-linked immunosorbent assay, nucleic acid hybridization assay, loop-mediated isothermal amplification assay, nucleic acid sequence based amplification, or reverse transcription polymerase chain reaction. In a further example, the method further includes discarding the second labeled sample.

In another example, a system for independently labeling partitioned microfluidic samples for identification includes a plurality of markers that are independently detectable relative to one another when present in an individual partitioned microfluidic sample and a microfluidic partition network to channel partitioned microfluidic samples. The system further includes a marker ingress network fluidically associated with the microfluidic partition network to introduce various combinations of the markers to individual partitioned microfluidic samples to allow for subsequent identification of the individual partitioned microfluidic samples based on a presence of a specific combination of the markers. In one example, the marker ingress network includes a microfluidic ejector, a microfluidic channel, an opening in a microfluidic channel, or a combination thereof. In another example, the microfluidic partition network, the microfluidic ingress network, or both are part of a lab on chip device.

In another example, a system for identifying an individual microfluidic sample from multiple partitioned microfluidic samples includes a microfluidic partition network to carry multiple partitioned microfluidic samples labeled with independent combinations of markers from a group of markers and a detection device to individually identify which markers from the group of markers that are present in individual partitioned microfluidic samples. In one example, the microfluidic partition network includes a multi-well chamber plate and a digital dispenser. In another example, the detection device includes a microfluidic chamber including a reference electrode, counter electrode, and working electrode. The working electrode is configured to detect the markers.

It is also noted that when discussing the method of labeling and identifying individual microfluidic samples, the system for independently labeling partitioned microfluidic samples for identification, or the system for identifying an individual microfluidic sample from multiple partitioned microfluidic samples such discussions of one example are to be considered applicable to the other examples, whether or not they are explicitly discussed in the context of that example. Thus, in discussing a combination of markers in the context of the method of labeling and identifying individual microfluidic samples, such disclosure is also relevant to and directly supported in the context of the system for independently labeling partitioned microfluidic samples for identification, the system for identifying an individual microfluidic sample from multiple partitioned microfluidic samples, and vice versa.

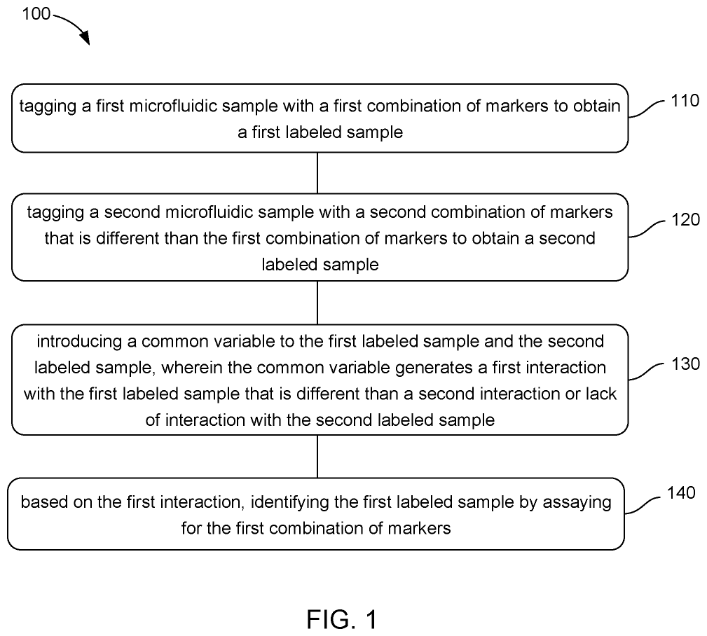

As illustrated in FIG. 1, a method 100 of labeling and identifying individual microfluidic samples can include tagging 110 a first microfluidic sample with a first combination of markers to obtain a first labeled sample and tagging 120 a second microfluidic sample with a second combination of markers that is different than the first combination of markers to obtain a second labeled sample. The method can further include introducing 130 a common variable to the first labeled sample and the second labeled sample. The common variable can generate a first interaction with the first labeled sample that is different than a second interaction or lack of interaction with the second labeled sample. Based on the first interaction, the method can further include identifying 140 the first labeled sample by assaying for the first combination of markers.

In further detail, the method can include tagging a first microfluidic sample with a first combination of markers and tagging a second microfluidic sample with a second combination of markers that is different from the first combination of markers. A combination of markers as used herein, can refer to 0, 1, 2, 3, 4, or more markers, up to an infinite amount. As used herein a combination of markers does not indicate that the combination always includes 2 or more markers and can include any quantity of markers so long as the first combination of markers differs from the second combination of markers. For example, a first combination of markers can exclude a marker and a second combination of markers can include one marker. In yet another example, a first combination of markers can include marker A and a second combination of markers can include markers A and B. A quantity of a combination of markers from a bank of possible markers can be determined using a permutation calculation. For example, a bank of 4 markers (A, B, C, and D) can be used to create the various combinations including, for example, A, B, C, D, AB, AC, AD, BC, BD, CD, ABC, ABD, BCD, and ABCD, as well as, a combination excluding, A, B, C, and D, e.g. without any markers. In one example, a bank of markers can provide from 4 to 128, from 6 to 128, from 8 to 128, or from 12 to 128 possible combinations of markers.

The individual markers in a combination of markers can include individual markers selected from a redox marker, a fluorescing chemical, a magnetic marker, an antigen, a fluorescent antigen, or a combination thereof. In one example, an individual marker can include a redox marker. In another example, an individual marker can include a fluorescing chemical. Examples of redox markers can include metal complexes of phenanthroline, metal complexes of bipyridine, methylene blue, nitrophenanthroline, n-phenylanthranilic acid, sodium diphenylamine sulfonate, and the like. Examples of fluorescing markers can include ferrocene, methylene blue, erythrosine extra bluish, diarylethene, stilbene, azobenzene, green fluorescent protein, quantum dots, nano-dots, and the like. Examples of magnetic markers can include magnetic isotopes, magnetic affinity beads, and the like. Examples of antigen markers can include affinity paring molecules such as antibodies with antigens, aptamers with antigens, and the like. Individual markers can be admixed in a carrier fluid. In one example, the carrier fluid can include water, phosphate buffer, saline, phosphate buffer saline, tris buffer, potassium acetate buffer, potassium phosphate buffer, phosphate buffered sucrose, hepes buffered saline, BES-buffered solution, Hanks balanced salt solution, or the like. In some examples, the carrier fluid can be selected based on the bioassay so that the carrier fluid does not interfere with the bioassay. The marker can be dispersed in the carrier fluid at from 0.01 wt % to 5 wt %. In other examples the marker can be dispersed in the carrier fluid at from 0.1 wt % to 3 wt %, at from 0.5 wt % to 2 wt %, or from 0.01 wt % to 4 wt %.

In one example, a tagging of a first microfluidic sample with the first combination of markers or tagging a second microfluidic sample with the second combination of markers can include admixing from 2 pL to 10 nL per individual marker dispersed in a carrier fluid into a fluidic volume of from 1 μL to 10 μL of the first microfluidic sample or the second microfluidic sample, respectively. In yet another the tagging can include admixing from 2 pL to 5 nL per individual marker dispersed in a carrier fluid into a fluidic volume of from 1 μL to 10 μL of the first microfluidic sample or the second microfluidic sample, respectively. The tagging can be repeated until the first microfluidic sample, the second microfluidic sample, or both are tagged with the desired combination of markers.

The admixing of the marker dispersed in a carrier fluid and the sample (the first microfluidic sample, the second microfluidic sample, or both) can occur manually or can occur using automated methods. In one example, the admixing can occur by dispensing an individual marker dispersed in a carrier fluid via pipette into the first microfluidic sample, the second microfluidic sample, or both. In another example, the admixing can occur by digitally dispensing an individual marker dispersed in a carrier fluid into the first microfluidic sample, the second microfluidic sample, or both. Digital dispensing can occur, for example, using a piezo-electric print head, a thermal ink-jet print head, a digital dispenser, and the like. In one example, the dispensing can occur into an 1 μL to 5 mL volume of the first microfluidic sample, the second microfluidic sample, or both. In another example, the dispensing can occur into a partitioned volume of the first microfluidic sample, the second microfluidic sample, or both. In one example, the partitioned volume can be from 300 μL to 400 μL. In one example, the partitioned volume can be located in an individual well of a well plate. In another example, the partitioned volume can be located in a channel of a microfluidic device.

In some examples, the method can further include individually dispensing fluidic blanks between partitioned volumes of the first microfluidic sample, the second microfluidic sample, or both when located in a microfluidic channel to provide separation between partitioned volumes of these samples. An example, of fluidic blanks can include oil, mineral oil, polydimethyl siloxane, hexadecane, isopar M, or the like and can be dispersed at volume to separate partitioned volumes from one another in a microfluidic channel.

Following tagging, the first labeled sample and the second labeled sample can be combined with one another. In some examples, the first labeled sample and the second labeled sample can be combined as multiple, distinct partitioned volumes, such as droplets. In one example, the first labeled sample, the second labeled sample, or both can be partitioned into multiple 3 pL to 1 μL partitioned volumes. In other examples, the first labeled sample, the second labeled sample, or both can be partitioned into multiple 3 pL to 20 pL volumes, 5 pL to 5,000 pL volumes, 500 pL to 10,000 pL volumes, or 1,000 pL to 100,000 pL volumes. In some examples, the first labeled sample, the second labeled sample, or both can be partitioned into droplets having a diameter of 20 μm by 20 μm by 20 μm.

The method can further include introducing a common variable to the combined first labeled sample and the second labeled sample. The common variable can include reactants for polymerase chain reaction, enzyme-linked immunosorbent assay, nucleic acid hybridization assay, loop-mediated isothermal amplification assay, nucleic acid sequence based amplification, or reverse transcription polymerase chain reaction. In one example, the common variable can interact with the first labeled sample and can result in a first interaction that is different than a second interaction or lack of interaction with the second labeled sample. In another example, the common variable can interact with portions of the first labeled sample and portions the second labeled sample and can lack an interaction with other portions of the first labeled sample and other portions of the second labeled sample.

A reaction can depend on the sample and the reactants. Introducing a common variable with partitioned volumes of the first labeled sample and the second labeled sample can permit absolute quantitation of reacting nucleic acids. In addition, introducing a common variable with partitioned volumes of the first labeled sample and the second labeled sample can permit for a large number of samples, from 2 to 65,000, to be simultaneously processed. Following a reaction or lack thereof, a combination of markers in the first labeled sample and the second labeled sample can be determined. Based on the combination of markers the identity of the source, i.e. the first microfluidic sample or the second microfluidic sample, can be determined.

In some examples, the method can further include discarding partitioned volumes that did not react with the common variable. Discarding partitioned volumes can reduce the time associated with quantifying positive reactions by limiting a quantity the partitioned volumes subject to the determination process. In another example, the method can further include discarding partitioned volumes that reacted with the common variable. This can allow for concentration of samples without components that would otherwise subtract from a further analysis.

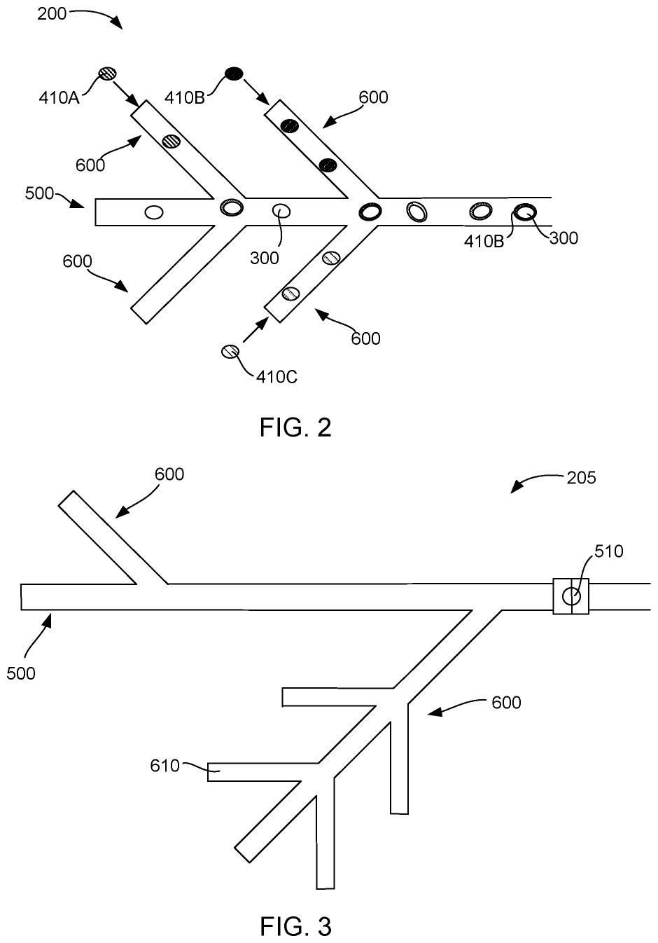

In further detail, as illustrated in FIG. 2, a system 200 for independently labeling partitioned microfluidic samples 300 for identification is shown. The system can include a plurality of markers 410A, 410B, and 410C, such as chemical markers, which can be independently detectable relative to one another when present in an individual partitioned microfluidic sample. The markers are shown in this FIG as structures associated with the partitioned microfluidic samples, but it is understood this is to illustrate the concept, and does not necessarily indicate that the marker is in the form of a coating or layer. In this example, three markers are shown, but the possible markers selected from in this example could be from a library of 3 markers, 4 markers, 5 markers, 6 markers, etc. The more possible markers that can be used, the greater the number of combinations that can be used to mark a sample, for example. In this example, a microfluidic partition network 500 can be used to channel partitioned microfluidic samples. A marker ingress network 600 (shown as four ingress branches in this example) can be fluidically associated with the microfluidic partition network to introduce various combinations of the markers to individual partitioned microfluidic samples to allow for subsequent identification of the individual partitioned microfluidic samples based on a presence of a specific combination of the markers. The plurality of markers can be as described above, with respect to the combination of markers. Thus, in this example there are four ingress branches, three of which are being used to introduce three different markers.

In one example, the microfluidic partition network and the marker ingress network can be part of the same device, as shown in FIGS. 2 and 3. Thus, both FIGS. 2 and 3 graphically illustrate a microfluidic partition network 500 that includes a microfluidic channel. The marker ingress network 600 can comprise injection ports that can be fluidly connected to the microfluidic channel of the microfluidic partition network. As shown in FIG. 2, the marker ingress network includes ingress branches that directly feed the microfluidic partition network. As shown at 205 in FIG. 3, however, the marker ingress network can itself included marker ingress channels that are branched with an injection port 610 (or even a series of injection ports as shown) that feed a channel that directly feeds the microfluidic partition network or partition channel thereof. In further detail as shown in FIG. 3 by example, the microfluidic partition network can further include an electrode 510 that can detect a change in an electric current passing thereby and can allow for the detection of the first labeled sample, the second labeled sample, or both as they pass through the microfluidic channel.

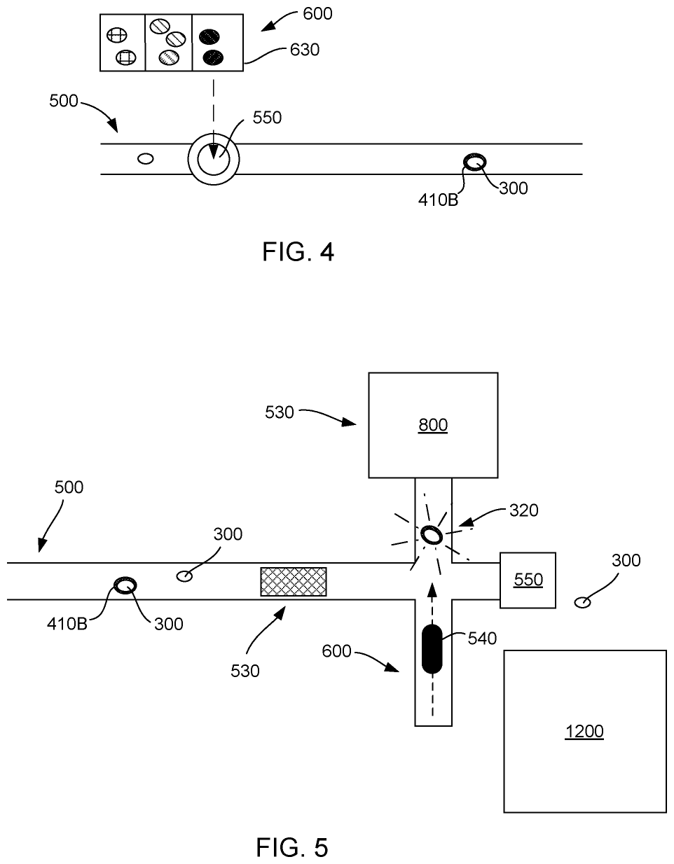

In yet another example, as shown by example in FIG. 4, a microfluidic partition network 500 and a marker ingress network 600 can be part of separate devices or separated components within the system. For example, the marker partition network can include a microfluidic channel with an opening 550 in an upward facing surface of the microfluidic channel, though this orientation is by example and other orientations can be designed. The marker ingress network can include a digital dispenser 630 that can introduce various combinations of markers 410B to a microfluidic sample 300 via the opening in the microfluidic channel as illustrated in FIG. 4.

In one example, and as shown in FIG. 5, a microfluidic partition channel 500 or a marker ingress network 600 or a combination of both can include a detector(s) 530. In this example, multiple detectors are shown, namely a CMOS photodiode detector 700 and a second detection device 800. Also in this example, there is a resistor 540 that can be used for sorting a partitioned volume of a sample 300 (with a marker 410B in one direction and without a marker in another direction). Thus, in one example, there may be detection that occurs at the CMOS photodiode detector. In another example, a marker providing a fluorescing sample 320 can be directed to the second detection device. As an example where both may work together, a partitioned volume that generates fluorescence upon interacting can be detected by the CMOS photodiode detector and subsequently trigger the resistor to fire, direct the fluorescing partitioned volume to the second detecting device. The second detecting device can include any type of sensor or assay device that may be applicable for screening the sample that is received. On the other hand, a partitioned volume that did not interact with the common variable, e.g., fluoresce in this example, can be discarded through an egress network 550 to a waste receptacle 1200.

With respect to the marker ingress network, an arrangement of the marker ingress network is not limited. The marker ingress network can include a combination of components. In one example, the marker ingress network can include a microfluidic ejector, a microfluidic channel, an opening in a microfluidic channel, or a combination thereof. The components of the marker ingress network can be part of a single device or can include components in multiple devices that can permit the introduction of various combinations of the markers to the fluid microchannel of the microfluidic partition network. In one example, the microfluidic partition network, the microfluidic ingress network, or both can be included as part of a lab on chip device.

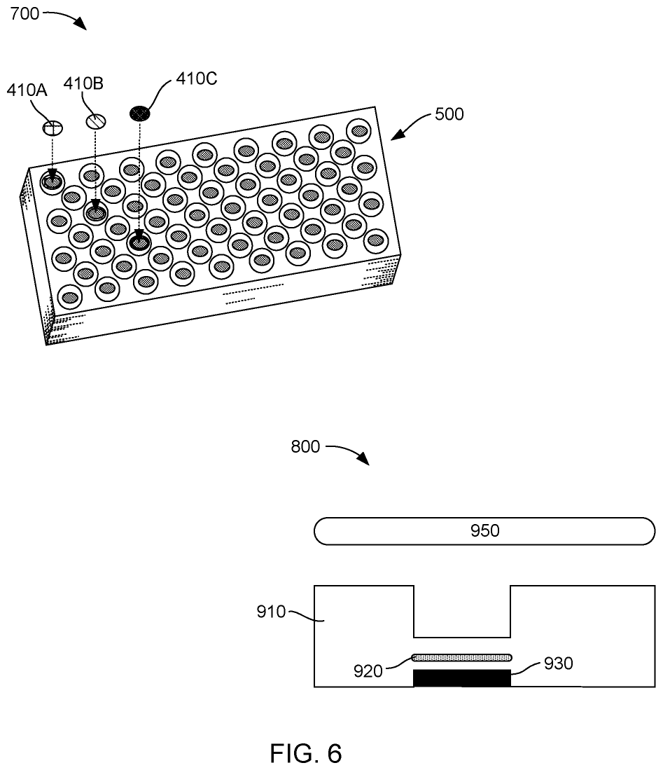

In another example, a system 700 is shown at FIG. 6 for identifying an individual microfluidic sample from multiple partitioned microfluidic samples. In one example, the system can include a microfluidic partition network 500 to carry multiple partitioned microfluidic samples labeled with independent combinations of markers (410A, 410B, and 410C) from a group of markers, e.g., chemical markers from a group of chemical markers. The microfluidic partition network in this example is in the form of a multi-well plate and may include a disperser, such as a digital dispenser, to eject fluid from the wells (or microchannels associated with the wells) to a detection device 800 to individually identify which markers from the group of markers are present in individual partitioned microfluidic samples. For example, the digital dispenser can be used to dispense partitioned volumes of the first sample fluid, the second sample, or both, as well as individual markers dispersed in carrier fluid. In another example, the microfluidic partition network can be as described above and/or as illustrated in FIGS. 2-5.

The detection device 800 can include a sensor or an arrangement of sensors with any of a number of sensors. In the example shown, the sensor is in the form of a photodiode 930 and an optical filter 920 embedded in a substrate 910, and further includes a light source 950. In other examples, the optical sensing system can include any of a number of combinations of the photodiode, the light source, a diffuse photo sensor, a specular photo sensor, an optical filter, a RGB mask, an LED, a confocal optic, and/or an internal reflector. An internal reflector can act to direct a light path to a photodiode and/or a photo sensor of the optical sensing system. In some examples, components of the optical sensing system can embedded in a single device. In another example, components of the optical sensing system can include a combination of on device components and off device components. In yet another example, the optical sensing system can include off device components. The illumination and collection of the light path can be in the same direction or they can be in different directions when considered with relation to one another.

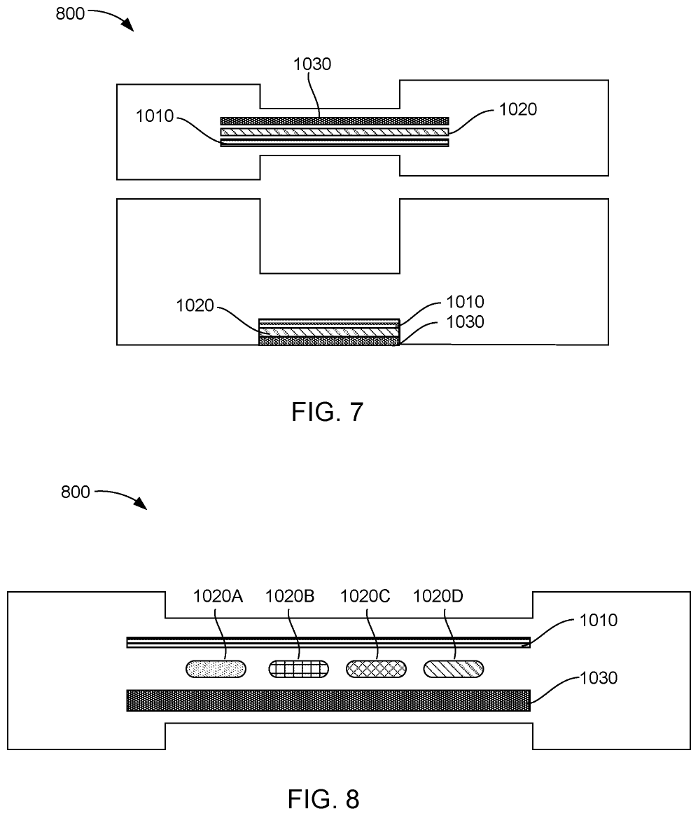

In one example, the detection device can be an electrochemical detection system, with examples shown in FIGS. 7 and 8. Thus, an electrochemical detection system can include a microfluidic chamber, a reference electrode, a counter electrode, and a working electrode. The working electrode can be configured to detect an electrical signal generated by individual markers in a combination of markers. The reference electrode can allow for the measurement of an electrochemical response generated at the working electrode. The reference electrode can be constructed to have a stable and known electrode potential, and can be used in a half-cell, or in some other cell configuration, to provide a reference for evaluating a material based on its electrochemical signature. In one example, the reference electrode can have stable electrical properties. The counter-electrode can close an electric circuit and balance a reaction occurring at the working electrode.

More specifically as shown in FIG. 7, an arrangement of a reference electrode, a counter electrode, and a working electrode can be provided, with specific arrangements varying. In some examples, the arrangement can include a “u-shaped” arrangement with the counter electrode and the reference electrode creating the u-shape and the working electrode can be disposed there between. In another example, the detection device can be as shown in FIG. 7, including a reference electrode 1010, a counter electrode 1030, and a working electrode 1020. In some examples, the counter electrode can be from 2 times to 10 times larger in area than the working electrode and the reference electrode.

In another example, the detection device can be as shown in FIG. 8 with a reference electrode 1010, a counter electrode 1030, and multiple working electrodes 1020A-1020D. The working electrodes can include a member of a molecule pair that specifically interacts with an individual marker. For example, the marker can include an antibody and the member of the molecule pair can include an antigen, or vice versa. In another example, the marker can include an aptamer and the member of the molecule pair can be an antigen, or vice versa. In a further example, marker can include a nucleic acid and the member of the molecule pair can include a complementary nucleic acid, or vice versa. Locating a member of the molecule pair at a known location can enable a readout at specific identifiable sensors and can detect a specific interaction that occurs.

In some examples, the detection device can be configured such that the counter electrode, the working electrode, or a both can include hydrophilic and lipophobic components. For example, the counter electrode, the working electrode, or a both can have a surface material that can include a glassy carbon, indium tin oxide, trans-cyclooctene, or a combination thereof. When the first labeled sample or the second labeled sample are exposed to a hydrophilic electrode, redox reagents in the sample can travel to the electrode surface and react. In one example, a chamber of the detection device can include hydrophobic walls and a hydrophilic floor which can cause the sample to open towards a hydrophilic electrode. In some examples, the electrochemical sensing can further include a mechanical squeezing element which can squish the sample and change a contact angle of the sample in relation to the counter electrode, the working electrode, or a both. An electric potential can be applied to the working electrode in a range that can vary from −5V to +5V, from −3V to +3V, or from −1V to +1V. In one example, the electric potential can be swept and a current detected at the working electrode can be recorded. An increase in current can indicate a presence of a marker in the combination of markers.

In yet another example, the detection device can include a magnetic detection system. A magnetic detection system can include a magnetic bead. In another example, a magnetic detection system can include a magnetoresistance element. The magnetoresistance element can be iron-chromium based, cobalt-copper based, cobalt-iron-copper based, or the like.

In some examples, the detection device can include a combination of detection systems. For example, the detection device can include an optical sensing system and an electrochemical detection system. In another example, the detection device can include an optical sensing system, and a magnetic detection system. In yet another example, the detection device can include an optical sensing system, an electrochemical detection system, and a magnetic detection system.

It is noted that, as used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though members of the list is individually identified as a separate and unique member. Thus, no individual member of such list should be construed as a de facto equivalent of any other member of the same list solely based on their presentation in a common group without indications to the contrary.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if numerical values and sub-ranges is explicitly recited. For example, a weight ratio range of 1 wt % to 20 wt % should be interpreted to include not only the explicitly recited limits of 1 wt % and 20 wt %, but also to include individual weights such as 2 wt %, 11 wt %, 14 wt %, and sub-ranges such as 10 wt % to 20 wt %, 5 wt % to 15 wt %, etc.

What is claimed is:

1. A method of labeling and identifying individual microfluidic samples, comprising:
tagging a first microfluidic sample with a first combination of markers to obtain a first labeled sample;
tagging a second microfluidic sample with a second combination of markers that is different than the first combination of markers to obtain a second labeled sample;
introducing a common variable to the first labeled sample and the second labeled sample, wherein the common variable generates a first interaction with the first labeled sample that is different than a second interaction or lack of interaction with the second labeled sample; and
based on the first interaction, identifying the first labeled sample by assaying for the first combination of markers.

2. The method of claim 1, wherein tagging first microfluidic sample with the first combination of markers includes admixing from 2 μL to 10 nL per individual marker into a fluidic volume from 1 μL to 10 μL of the first microfluidic sample.

3. The method of claim 1, wherein a marker in the first combination of markers and the second combination of markers include individual markers selected from a redox marker, a fluorescing chemical, a magnetic marker, an antigen, a fluorescent antigen, phosphorescent, chemiluminescent, or a combination thereof.

4. The method of claim 1, wherein the first combination of markers and the second combination of markers are selected from a bank of markers that are independently identifiable relative to one another when present in the first labeled sample or the second labeled sample, and the bank of markers provide for combinations of markers generated from 4 to 128 possible combinations of markers.

5. The method of claim 1, the first labeled sample, the second labeled sample, or both are partitioned into multiple 3 pL to 1 μL partitioned volumes prior to the introducing of the common variable to individual partitioned volumes.

6. The method of claim 5, further comprising individually dispensing the partitioned volumes into individual wells of a well plate or individual channels of a microfluidic device either before or after introducing the common variable to the first labeled sample and the second labeled sample.

7. The method of claim 5, further comprising individually dispensing the partitioned volumes into a microfluidic device with fluidic blanks providing separation between partitioned volumes.

8. The method of claim 1, wherein the common variable includes reactants for polymerase chain reaction, enzyme-linked immunosorbent assay, nucleic acid hybridization assay, loop-mediated isothermal amplification assay, nucleic acid sequence based amplification, or reverse transcription polymerase chain reaction.

9. The method of claim 1, further comprising discarding the second labeled sample.

10. A system for independently labeling partitioned microfluidic samples for identification, comprising:
a plurality of markers that are independently detectable relative to one another when present in an individual partitioned microfluidic sample;
a microfluidic partition network to channel partitioned microfluidic samples; and
a marker ingress network fluidically associated with the microfluidic partition network to introduce various combinations of the markers to individual partitioned microfluidic samples to allow for subsequent identification of the individual partitioned microfluidic samples based on a presence of a specific combination of the markers.

11. The system of claim 10, wherein the marker ingress network includes a microfluidic ejector, a microfluidic channel, an opening in a microfluidic channel, or a combination thereof.

12. The system of claim 10, wherein the microfluidic partition network, the microfluidic ingress network, or both are part of a lab on chip device.

13. A system for identifying an individual microfluidic sample from multiple partitioned microfluidic samples, comprising:
a microfluidic partition network to carry multiple partitioned microfluidic samples labeled with independent combinations of markers from a group of markers; and
a detection device to individually identify which markers from the group of markers that are present in individual partitioned microfluidic samples.

14. The system of claim 13, wherein the microfluidic partition network includes a multi-well chamber plate and a digital dispenser.

15. The system of claim 13, wherein the detection device comprises a microfluidic chamber including a reference electrode, counter electrode, and working electrode, and wherein the working electrode is configured to detect the markers.
